(19) United States
(12) Patent Application Publication
(10) Pub. No.: US 2007/0249556 A1
(43) Pub. Date: Oct. 25, 2007

(54) METHOD OF TREATING INFLAMMATION

(76) Inventors: Kurt E. Brubaker, Durham, NC (US); Sammy R. Shaver, Chapel Hill, NC (US); James G. Douglass, Apex, NC (US)

Correspondence Address: HOWERY LLP, C/O IP DOCKETING DEPARTMENT, 2941 FAIRVIEW PARK DRIVE SUITE 200, FALLS CHURCH, VA, 22042, US

(21) Appl. No.: 11/737,633

(22) Filed: Apr. 19, 2007

Related U.S. Application Data
(60) Provisional application No. 60/793,949, filed on Apr. 21, 2006.

(51) Int. Cl. A61K 31/7076

(52) U.S. Cl. 514/47

(57) ABSTRACT

This invention provides methods of preventing and/or treating diseases or conditions associated with inflammation in a mammal, particularly a human. The method comprises administering to a mammal in need thereof an effective amount of a compound of Formula I, IA, or IB, wherein said amount is effective to inhibit inflammation. The invention also provides methods for inhibiting chemotaxis of leukocytes.

This application claims priority to U.S. provisional application No. 60/793,949, filed Apr. 21, 2006. The content of which is incorporated herein by reference in its entirety.

### TECHNICAL FIELD

This invention relates to a method of modulating inflammatory cell migration. More particularly, the present invention relates to a method of treating diseases or conditions in patients with harmful inflammation resulting from aberrant inflammatory cell migration.

### BACKGROUND OF THE INVENTION

Inflammation is a reaction to cellular injury that typically includes blood vessel dilation, leukocyte (neutrophils, eosinophils, lymphocytes, monocytes, basophils, macrophages, dendritic cells, and mast cells) infiltration, redness, pain and swelling, called the inflammatory response. The inflammatory response serves the purpose of eliminating harmful agents from the body. There is a wide range of insults that can initiate an inflammatory response including infection, allergens, autoimmune stimuli, immune response to transplanted tissue, toxins, ischemia/reperfusion, hypoxia, and mechanical or thermal trauma. The body's response becomes an agent of disease when inflammation results in inappropriate injury to host tissues in the process of eliminating the targeted agent, or responding to a traumatic insult (see Linden et al, U.S. Pat. No. 6,232,297).

Neutrophils are a subset of leukocytes that comprise an essential component of the host defense system against microbial invasion. In response to soluble pro-inflammatory mediators released by cells at the site of injury, neutrophils migrate into tissue from the bloodstream by crossing the blood vessel wall. At the site of injury, activated neutrophils kill foreign cells by phagocytosis and by the release of cytotoxic compounds, such as oxidants, proteases and cytokines. Despite their importance in fighting infection, neutrophils themselves can promote tissue damage. During an abnormal inflammatory response, neutrophils can cause significant tissue damage (or cell death) by releasing toxic substances at the vascular wall or in uninjured tissue, which are intended to kill foreign cells but once released do not discriminate and can kill host cells as well. Alternatively, neutrophils that stick to the capillary wall or clump in venules may produce tissue damage by ischemia. Such abnormal inflammatory responses have been implicated in the pathogenesis of a variety of clinical disorders including: adult respiratory distress syndrome (ARDS), ischemia-reperfusion injury (following myocardial infarction, shock, stroke, and organ transplantation), acute and chronic allograft rejection, vasculitis, sepsis, rheumatoid arthritis, and inflammatory skin diseases (Carlos, T. M., et al., 1990 Immunol. Rev. 114, 5).

An increased presence of inflammatory cells, or leukocytes involved in the inflammatory response, are characteristic features of a number of respiratory diseases including chronic obstructive respiratory disease (COPD), cystic fibrosis and some subsets of patients with asthma (Barnes, P. J., 2007, J. of Allergy and Clinical Immunology, article in press). The presence of increased numbers of leukocytes, particularly neutrophils, is thought to play an important role in respiratory disease pathogenesis.

The release of inflammatory cytokines such as tumor necrosis factor-alpha (TNF-α) by leukocytes is a means by which the immune system combats pathogenic invasions, including infections. TNF-α stimulates the expression and activation of adherence factors on leukocytes and endothelial cells, primes neutrophils for an enhanced inflammatory response to secondary stimuli and enhances adherent neutrophil oxidative activity. In addition, macrophages/dendritic cells act as accessory cells processing antigen for presentation to lymphocytes. The lymphocytes, in turn, become stimulated to act as pro-inflammatory cytotoxic cells.

Generally, cytokines stimulate neutrophils to enhance oxidative (e.g., superoxide and secondary products) and nonoxidative (e.g., myeloperoxidase and other enzymes) inflammatory activity. Inappropriate and over-release of cytokines can produce counterproductive exaggerated pathogenic effects through the release of tissue-damaging oxidative and nonoxidative products. For example, TNF-α can induce neutrophils to adhere to the blood vessel wall and then to migrate through the vessel to the site of injury and release their oxidative and non-oxidative inflammatory products. This normal component of the inflammatory response can be toxic to the host cells if inappropriately high concentrations of TNF-α are released.

The mechanism by which leukocytes leave the bloodstream and accumulate at inflammatory sites involves three distinct steps: (1) rolling, (2) arrest and firm adhesion, and (3) transendothelial migration (Wagner, J. G., et al., Pharm. Rev. 52:349-374, 2000). The second step is mediated at the molecular level by chemoattractant receptors on the surface of leukocytes which bind chemoattractant cytokines secreted by proinflammatory cells at the site of damage or infection. Receptor binding activates leukocytes, increases their adhesiveness to the endothelium, and promotes their transmigration into the affected tissue, where they can secrete inflammatory and chemoattractant cytokines and degradative proteases that act on the subendothelial matrix, facilitating the migration of additional leukocytes to the site of injury (see Laborde et al, U.S. Pat. No. 6,809,113, issued Oct. 26, 2004). Specific molecules such as fMLP (Wagner, J. G., Pharm. Rev. 52:349-374, 2000), and bradykinin (Gouget, J. et al, JPET 309:661-669, 2004), have been shown to exert chemoattractant effects on neutrophils.

While significant efforts have been made to utilize inhibitors of pro-inflammatory mediator signaling, there exists a need to find efficacious methods of modulating inflammatory conditions with acceptable safety profiles.

### SUMMARY OF THE INVENTION

The present invention provides methods for preventing and/or treating diseases or conditions associated with inflammation in a mammal, particularly a human. The method comprises administering to a mammal in need thereof an effective amount of a compound of Formula I, wherein said amount is effective to inhibit inflammation.

The present invention also provides methods of inhibiting cellular chemotaxis, such as leukocyte (e.g., neutrophil) chemotaxis. The methods comprise contacting white blood cells, with one or more Formula I compound, at an effective concentration to inhibit chemotaxis of white blood cells.

### BRIEF DESCRIPTION OF THE FIGURES

FIG. 1 depicts a significant reduction in the number of neutrophils found in the lumen of the lungs of rats treated with Compound 11 compared with animals not receiving Compound 11 in an acute model of pulmonary inflammation.

### DETAILED DESCRIPTION OF THE INVENTION

### Definitions

When present, unless otherwise specified, the following terms are generally defined as, but are not limited to, the following:

Halo substituents are taken from fluorine, chlorine, bromine, and iodine.

Alkyl groups are from 1 to 12 carbon atoms inclusively, either straight chained or branched, are more preferably from 1 to 8 carbon atoms inclusively, and most preferably 1 to 6 carbon atoms inclusively.

Alkylene chains are from 2 to 20 carbon atoms inclusively, have two points of attachment to the to the molecule to which they belong, are either straight chained or branched, can contain one or more double and/or triple bonds, are more preferably from 4 to 18 atoms inclusively, and are most preferably from 6 to 14 atoms inclusively.

Alkenyl groups are from 1 to 12 carbon atoms inclusively, either straight or branched containing at least one double bond but can contain more than one double bond.

Alkynyl groups are from 2 to 12 carbon atoms inclusively, either straight or branched containing at least one triple bond but can contain more than one triple bond, and additionally can contain one or more double bonded moieties.

“Alkoxy” refers to the group alkyl-O— wherein the alkyl group is as defined above including optionally substituted alkyl groups as also defined above.

“Aryl” refers to an unsaturated aromatic carbocyclic group of from 6 to 14 carbon atoms inclusively having a single ring (e.g., phenyl) or multiple condensed rings (e.g., naphthyl or anthryl). Preferred aryls include phenyl, naphthyl and the like.

“Arylalkyl” refers to aryl-alkyl- groups preferably having from 1 to 6 carbon atoms inclusively in the alkyl moiety and from 6 to 10 carbon atoms inclusively in the aryl moiety. Such arylalkyl groups are exemplified by benzyl, phenethyl and the like.

“Arylalkenyl” refers to aryl-alkenyl- groups preferably having from 1 to 6 carbon atoms in the alkenyl moiety and from 6 to 10 carbon atoms inclusively in the aryl moiety.

“Arylalkynyl” refers to aryl-alkynyl- groups preferably having from 1 to 6 carbon atoms inclusively in the alkynyl moiety and from 6 to 10 carbon atoms inclusively in the aryl moiety.

“Aryloxy” refers to the group aryl-O— wherein the aryl group is as defined above including optionally substituted aryl groups as also defined above.

“Cycloalkyl” refers to cyclic alkyl groups of from 3 to 12 carbon atoms inclusively having a single cyclic ring or multiple condensed rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantyl, and the like.

“Cycloalkenyl” refers to cyclic alkenyl groups of from 4 to 12 carbon atoms inclusively having a single cyclic ring or multiple condensed rings and at least one point of internal unsaturation, which can be optionally substituted with from 1 to 3 alkyl groups. Examples of suitable cycloalkenyl groups include, for instance, cyclobut-2-enyl, cyclopent-3-enyl, cyclooct-3-enyl and the like.

“Cycloalkylalkyl” refers to cycloalkyl-alkyl- groups preferably having from 1 to 6 carbon atoms inclusively in the alkyl moiety and from 6 to 10 carbon atoms inclusively in the cycloalkyl moiety. Such cycloalkylalkyl groups are exemplified by cyclopropylmethyl, cyclohexylethyl and the like.

“Heteroaryl” refers to a monovalent aromatic carbocyclic group of from 1 to 10 carbon atoms inclusively and 1 to 4 heteroatoms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to heteroaryl-alkyl- groups preferably having from 1 to 6 carbon atoms inclusively in the alkyl moiety and from 6 to 10 carbon atoms inclusively in the heteroaryl moiety. Such arylalkyl groups are exemplified by pyridylmethyl and the like.

“Heteroarylalkenyl” refers to heteroaryl-alkenyl- groups preferably having from 1 to 6 carbon atoms inclusively in the alkenyl moiety and from 6 to 10 carbon atoms inclusively in the heteroaryl moiety.

“Heteroarylalkynyl” refers to heteroaryl-alkynyl- groups preferably having from 1 to 6 carbon atoms inclusively in the alkynyl moiety and from 6 to 10 carbon atoms inclusively in the heteroaryl moiety.

“Heterocycle” refers to a single ring or multiple condensed rings, from 1 to 8 carbon atoms inclusively and from 1 to 4 hetero atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Preferred heterocycles include piperidinyl, pyrrolidinyl and tetrahydrofuryl.

Examples of heterocycles and heteroaryls include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

Positions occupied by hydrogen in the foregoing groups can be further substituted with substituents exemplified by, but not limited to, hydroxy, oxo, nitro, methoxy, ethoxy, alkoxy, substituted alkoxy, fluoro, chloro, bromo, iodo, methyl, ethyl, propyl, butyl, alkyl, substituted alkyl, thio, thioalkyl, acyl, carboxyl, alkoxycarbonyl, carboxamido, substituted carboxamido, alkylsulfonyl, alkylsulfinyl, alkylsulfonylamino, sulfonamido, substituted sulfonamide, cyano, amino, substituted amino, acylamino, trifluoromethyl, trifluoromethoxy, phenyl, aryl, substituted aryl, pyridyl, imidazolyl, heteroaryl, substituted heteroaryl, cyclopropyl, cyclobutyl, cyclopentyl, cyclohexyl, cycloalkyl, substituted cycloalkyl, pyrrolidinyl, piperidinyl, morpholino, and heterocycle; and preferred heteroatoms are oxygen, nitrogen, and sulfur. It is understood that where open valences exist on these substituents they can be further substituted with alkyl, cycloalkyl, aryl, heteroaryl, and/or heterocycle groups, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

Pharmaceutically acceptable salts are salts that retain the desired biological activity of the parent compound and do not impart undesired toxicological effects. Pharmaceutically acceptable salt forms include various polymorphs as well as the amorphous form of the different salts derived from acid or base additions. The acid addition salts can be formed with inorganic or organic acids. Illustrative but not restrictive examples of such acids include hydrochloric, hydrobromic, sulfuric, phosphoric, citric, acetic, propionic, benzoic, napthoic, oxalic, succinic, maleic, malic, adipic, lactic, tartaric, salicylic, methanesulfonic, 2-hydroxyethanesulfonic, toluenesulfonic, benzenesulfonic, camphorsulfonic, and ethanesulfonic acids. The pharmaceutically acceptable base addition salts can be formed with metal or organic counterions and include, but are not limited to, alkali metal salts such as sodium or potassium; alkaline earth metal salts such as magnesium or calcium; and ammonium or tetraalkyl ammonium salts, i.e., NX₄⁺ (wherein X is C₁₋₄).

Tautomers are compounds that can exist in one or more forms, called tautomeric forms, which can interconvert by way of a migration of one or more hydrogen atoms in the compound accompanied by a rearrangement in the position of adjacent double bonds. These tautomeric forms are in equilibrium with each other, and the position of this equilibrium will depend on the exact nature of the physical state of the compound. It is understood that where tautomeric forms are possible, the current invention relates to all possible tautomeric forms.

Solvates are addition complexes in which a compound of Formula I or II is combined with a pharmaceutically acceptable cosolvent in some fixed proportion. Cosolvents include, but are not limited to, water, methanol, ethanol, 1-propanol, isopropanol, 1-butanol, isobutanol, tert-butanol, acetone, methyl ethyl ketone, acetonitrile, ethyl acetate, benzene, toulene, xylene(s), ethylene glycol, dichloromethane, 1,2-dichloroethane, N-methylformamide, N,N-dimethylformamide, N-methylacetamide, pyridine, dioxane, and diethyl ether. Hydrates are solvates in which the cosolvent is water. It is to be understood that the definition of compounds in Formulae I and II encompasses all possible hydrates and solvates, in any proportion, which possess the stated activity.

“Chemotaxis” refers to the directional migration of cells in response to a chemical concentration gradient. “Chemotaxis” as used herein, refers to positive chemotaxis, which is migration of cells towards a higher concentration.

“Neutrophil” refers to the most numerous polymorphonuclear leukocyte found in the blood; a phagocytic cell of the myeloid series that is distinguished by the presence of cytoplasmic azurophil granules and other granules that take up neither acidic nor basic dyes. It plays a major role in the inflammatory response, undergoing chemotaxis towards sites of infection or wounding.

The inventors have discovered particular compounds that are effective on (a) inhibiting chemotaxis of leukocytes, particularly neutrophils, in vivo and in vitro, and/or (b) preventing and/or treating inflammation in a mammal. The compounds have a general structure of Formula I, preferably Formula IA or IB.

The invention provides methods of inhibiting cellular chemotaxis, preferably leukocyte (e.g., neutrophil) chemotaxis. The method comprises contacting white blood cells, particularly mammal white blood cells, especially human white blood cells, with one or more Formula I compound, at an effective concentration to inhibit chemotaxis of white blood cells.

The invention also provides methods of preventing and/or treating diseases or conditions associated with inflammation. The method comprises administering to a mammal, which is in need of anti-inflammation prevention or which suffers from inflammation, an effective amount of a compound of Formula I, wherein said amount is effective to inhibit inflammation. An effective amount is meant an amount effective to yield a sufficient plasma concentration of the compound or its active metabolite to inhibit chemotaxis of neutrophil towards the inflammation sites.

The present invention is useful in preventing and/or treating inflammation in mammals such as humans, domesticated companion animals (pets) or livestock animals.

Formula I compounds useful for this invention include the tautomers thereof, and/or pharmaceutically-acceptable hydrates, solvates, and/or salts thereof. Optionally, Formula I compounds can be used in combination with other compounds useful for the treatment of inflammatory disorders or diseases.

The inflammatory responses, which can be treated with a compound of Formula I, include pulmonary inflammation due to respiratory diseases such as chronic obstructive pulmonary disease (COPD), cystic fibrosis, and asthma. Chronic obstructive pulmonary disease is comprised primarily of two related diseases—chronic bronchitis and emphysema. In both diseases, there is chronic obstruction of the flow of air through the airways and out of the lungs, and the obstruction generally is permanent and progressive over time.

Other inflammatory conditions or diseases which can be treated with a compound of Formula I are:

(a) autoimmune stimulation (autoimmune diseases), such as lupus erythematosus, multiple sclerosis, infertility from endometriosis, type I diabetes mellitus including the destruction of pancreatic islets leading to diabetes and the inflammatory consequences of diabetes, including leg ulcers, Crohn's disease, ulcerative colitis, inflammatory bowel disease, osteoporosis and rheumatoid arthritis;

(b) Ocular inflammation associated with corneal ulcers, giant papillary conjunctivitis, blepharitis, chelazion, uveitis, dry eye, post-surgical inflammation, and contact lens associated inflammation

(c) allergic diseases such as hay fever, rhinitis, seasonal allergic conjunctivitis, vernal conjunctivitis and other eosinophil-mediated conditions;

(d) skin diseases such as psoriasis, contact dermatitis, eczema, infectious skin ulcers, open wounds, and cellulitis;

(e) infectious diseases including sepsis, septic shock, encephalitis, infectious arthritis, endotoxic shock, gram negative shock, Jarisch-Herxheimer reaction, shingles, toxic shock, cerebral malaria, bacterial meningitis, acute respiratory distress syndrome (ARDS), lyme disease, and HIV infection,

(f) wasting diseases such as cachexia secondary to cancer and HIV;

(g) inflammation due to organ, tissue or cell transplantation (e.g., bone marrow, cornea, kidney, lung, liver, heart, skin, pancreatic islets) including transplant rejection, and graft versus host disease;

(h) adverse effects from drug therapy, including adverse effects from amphotericin B treatment, adverse effects from immunosuppressive therapy, e.g., interleukin-2 treatment, adverse effects from OKT3 treatment, adverse effects from GM-CSF treatment, adverse effects of cyclosporine treatment, and adverse effects of aminoglycoside treatment, stomatitis, and mucositis due to immunosuppression;

(i) cardiovascular conditions including circulatory diseases induced or exasperated by an inflammatory response, such as ischemia, atherosclerosis, peripheral vascular disease, restenosis following angioplasty, inflammatory aortic aneurysm, vasculitis, stroke, spinal cord injury, congestive heart failure, hemorrhagic shock, ischemia/reperfusion injury, vasospasm following subarachnoid hemorrhage, vasospasm following cerebrovascular accident, pleuritis, pericarditis, and the cardiovascular complications of diabetes;

(j) dialysis, including pericarditis, due to peritoneal dialysis;

(k) gout; and

(l) chemical or thermal-induced inflammation due to burns, acid, alkali and the like.

### Formula I Compounds

Formula I compounds useful in the present invention include compounds of general Formula I, and/or tautomers thereof, and/or pharmaceutically-acceptable hydrates, solvates, and/or salts thereof:

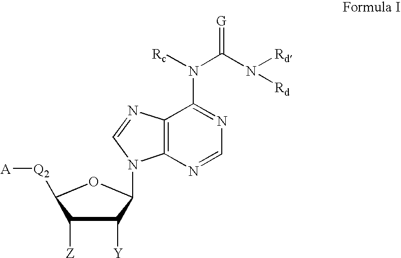

wherein;

Q₂=C₁₋₈ alkyl, C₂₋₈ alkenyl, C₂₋₈ alkynyl, optionally containing one or more heteroatoms, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ hetero cycle, —(CO)—, or absent,

A is H, —C₁₋₃ alkyl, C₂₋₃ alkenyl, C₂₋₃ alkynyl, and each hydrogen of said alkyl or alkenyl is optionally substituted by 0 to 2 fluorine groups, 0 to 1 methyl group, 0 to 2-[(CO)OR] groups, or 0 to 1 —(OR) group; (for example, A can be —CH₂—CH₂—CH₂F or —CH₂—CH(COOCH₃)—CH₃ or —CH(CH₃)—CH₂—CH₂OCH₃ or permutations of these), or

A is selected from the group consisting of H, —OR, —COOR, —SR, —S(O)L, —S(O₂)L, —SO₃H, —S(O₂)NRR, —S(O₂)NR(CO)L, —NRR, —NR(CO)L, —N[(CO)L]₂, —NR(SO₂)L, —NR(CO)NR(SO₂)L, —NR(SO₂)NRR, or —NR(SO₂)NR(CO)L; wherein each R and L is independently as defined below;

wherein the R groups of a —NRR unit (N,N-disubstituted-amino- group) in A optionally are taken together such that a ring of 3 to 7 members is formed, with or without heteroatoms in place of the ring-carbon units;

with the proviso that when A=H, then at least one of Rₐ or R_{b} is H; or

A is defined as in Formula II;

the first atom of the moiety A/Q₂ directly attached to the 4′ position is C;

Y=H, OH, or ORₐ;

Z=H, OH, or OR_{b}; with the proviso that Y and Z are not both H;

R is selected from the group consisting of: H, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ heterocycle; where all rings or chains optionally bear one or more desired substituents;

Rₐ and R_{b} are residues which are linked directly to the 2′ and/or 3′ oxygens of the furanose via a carbon atom according to Formula III, or linked directly to the two 2′ and 3′ oxygens of the furanose via a common carbon atom according to Formula IV;

R_{c}=H, OR, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ heterocycle, or L(CO)—;

L is selected from the group consisting of: H, —CF₃, —CF₂CF₃, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ heterocycle, C₁₋₆ alkoxy, aralkoxy, aryloxy, N,N-disubstituted-amino, N-substituted-amino, or unsubstituted-amino; where all rings or chains optionally bear one or more desired substituents; or

when L is N-substituted-amino, or N,N-disubstituted-amino, each substituent of said amino group of L is selected from the group consisting of: C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ heterocycle;

when L is N,N-disubstituted-amino, the two substituents are optionally taken together to form a ring of 3 to 7 members, wherein said formed ring thereon bears the remaining features of said selected substituents before said ring formation;

G=O, S or NR_{d} where R_{d} is defined as below;

R_{d} and R_{d′} are independently selected from the group consisting of: H, —CN, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ heterocycle;

with the proviso that no more than one cyano group is present in any (RC)—N⁶-(G)—NR_{d}R_{d′} unit; or

R_{d} and R_{d′} groups are taken together to form a ring of 4 to 7 members, with or without unsaturation and with or without heteroatoms in place of ring-carbon units; or

R_{d} or R_{d′} and R_{c} are taken together to form a ring of 4 to 7 members, with or without unsaturation and with or without heteroatoms in place of ring-carbon units; and with the further proviso that no compound in Formula I contains: a halogen- group, hydroxy- group, sulfhydryl- group, or amino- group (—NH₂, N-substituted-amino, or N,N-disubstituted-amino) attached to an sp³-hybridized-carbon atom that is bonded directly to a heteroatom selected from the group consisting of O, S, and N, as compounds in this class (e.g., —[C(OH)(SR)]—, —[CCl(NRR)]—, etc.) are in general of lower chemical stability; the first exception to this proviso is: compounds in which the said sp³-hybridized-carbon atom is bonded directly to: 1) a sulfur atom which is part of a —[S(O)]- group (sulfinyl group), or a —[S(O₂)]- group (sulfonyl group) and also to: 2) one or more halogen atoms (an example of a moiety having this arrangement is a trifluoromethanesulfonyl group); the second and final exception to this proviso is the C-1′ position of the furanose of compounds of Formula I where the sp³-hybridized-carbon atom at the 1′-position is attached to: 1) the oxygen atom of the furanose ring and to: 2) the nitrogen atom of the adenine;

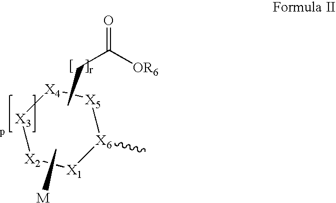

wherein:

X₆ is the attachment point to the moiety defined by Q₂;

the ring defined by X₁-X₆ is taken to mean a ring with or without unsaturation;

X₁-X₆ are independently C, N, O, or S; and

when any of X₁-Xs are C, the carbon atom is either unsubstituted (nothing is attached), or the carbon atom bears a variety of substituents such as halogen, alkyl, alkoxy, aminoalkyl, and the like; and

when any of X₁-X₅ is N in an saturated ring, the nitrogen atom is optionally bears substituents such as alkyl or acyl; or

any of X₁-X₅ is absent, with the proviso that at least two of X₁-X₅ are present, such that the ring described by X₁-X₆ consists of at least three atoms;

with the provisos that no two adjacent atoms X₁-X₆ are both O or S, and that the ring shown in Formula II contains no more than four heteroatoms, and that the shown pendant —CO₂R₇ unit in Formula II is a substituent on the ring described in Formula II, and that the ring of Formula II contains no halogen- group, hydroxy- group, sulfhydryl- group, or amino- group (—NH₂, N-substituted-amino, or N,N-disubstituted-amino) attached to an sp³-hybridized-carbon atom that is bonded directly to a heteroatom selected from the group consisting of O, S, and N, as such types of compounds are unstable in many cases;

p=0, 1, or 2;

r=0 or 1;

R₆ is H, a physiologically-relevant cation forming a carboxylate salt, alkyl, aryl, or aralkyl, with the resultant moiety C(═O)OR₆ preferably having an adjacent relationship to the attachment point of Q₂;

M is H, F, Cl, alkyl, or alkoxy;

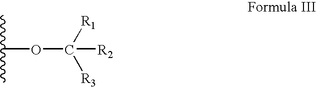

wherein:

O is the corresponding 2′ and/or 3′ oxygen of the furanose;

R₁, R₂, and R₃ are independently H, alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted, such that the moiety defined by Formula III is an ether; or

R₁ and R₂ are independently H, alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted; and R₃ is alkoxy, cycloalkoxy, aralkyloxy, aryloxy, substituted aralkyloxy, or substituted aryloxy, such that the moiety defined by Formula III is an acyclic acetal or ketal; or

R₁ and R₂ are taken together as oxygen or sulfur doubly bonded to C₁ and R₃ is alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted, such that the moiety defined by Formula III is an ester or thioester; or

R₁ and R₂ are taken together as oxygen or sulfur doubly bonded to C₁ and R₃ is amino or mono- or disubstituted amino, where the substituents are independently alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted, such that the moiety defined by Formula III is a carbamate or thiocarbamate; or

R₁ and R₂ are taken together as oxygen or sulfur doubly bonded to C₁ and R₃ is alkoxy, cycloalkoxy, aralkyloxy, aryloxy, substituted aralkyloxy, or substituted aryloxy, such that the moiety defined by Formula III is a carbonate or thiocarbonate; or

### R₃ is not present and R₁ and R₂ are taken together as oxygen or sulfur doubly bonded to C and both the 2′ and 3′ oxygens of the furanose are directly bound to C to form a cyclical carbonate or thiocarbonate;

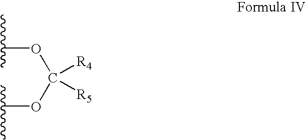

wherein 0 is the 2′ and 3′ oxygens of the furanose; and the 2′ and 3′ oxygens of the furanose are linked by a common carbon atom (C) to form a cyclical acetal, cyclical ketal, or cyclical orthoester;

for the cyclical acetal and ketal, R₄ and R₅ are independently hydrogen, alkyl, alkenyl, alkynyl, cycloalkyl, cycloalkenyl, aryl, aralkyl, aralkenyl, aralkynyl, heteroaryl, heteroarylalkenyl, heteroarylalkynyl, optionally substituted; or

R₄ and R₅ are joined together to form a homocyclic or heterocyclic ring composed of 3 to 8 atoms, preferably 3 to 6 atoms;

for the cyclical orthoester, R₄ is hydrogen, alkyl, alkenyl, alkynyl, cycloalkyl, cycloalkenyl, aryl, aralkyl, aralkenyl, aralkynyl, heteroaryl, heteroarylalkenyl, heteroarylalkynyl, optionally substituted, and R₅ is alkyloxy, cycloalkyloxy, aralkyloxy, aryloxy, substituted aralkyloxy, or substituted aryloxy.

One preferred subset of Formula I compounds are those Formula IA compounds, where Y, Z are defined above in Formula I;

Q₂=C₁₋₈ alkyl, C₂₋₈ alkenyl, C₂₋₈ alkynyl, optionally containing one or more heteroatoms, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ heterocycle, —(CO)—, or absent;

A is H, —OR, —SR, —C₁₋₃ alkyl, C₂₋₃ alkenyl, or C₂₋₃ alkynyl, and each hydrogen of said alkyl or alkenyl is optionally substituted by 0 to 2 fluorine groups, 0 to 1 methyl group, 0 to 2-[(CO)OR] groups, or 0 to 1 —(OR) group; for example, A can be —CH₂—CH₂—CH₂F or —CH₂—CH(COOCH₃)—CH₃ or —CH(CH₃)—CH₂—CH₂OCH₃ or pennutations of these;

R_{c}=H;

G=O;

R, R_{d} and R_{d′} are independently selected from the group consisting of: H, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, and C₂₋₆ heterocycle, where all rings or chains optionally bear one or more desired substituents; or

R_{d} and R_{d′} groups are taken together to form a ring of 4 to 7 members, with or without unsaturation and with or without heteroatoms in place of ring-carbon units.

Another preferred subset of compounds of Formula I fall under the definition of Formula IB:

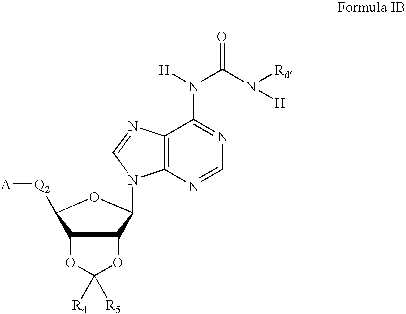

wherein Q₂=C₁₋₈ alkyl, C₂₋₈ alkenyl, C₂₋₈ alkynyl, optionally containing one or more heteroatoms, aralkyl, aralkenyl, aralkynyl, heteroaryl, heteroarylalkenyl, heteroarylalkynyl, C₂₋₆ heterocycle, or absent,

A is —OR, —SR, or —COOR;

the first atom of the moiety A/Q₂ directly attached to the 4′ position is C;

R and R_{d′} are independently selected from the group consisting of: H, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ heterocycle; where all rings or chains optionally bear one or more desired substituents; and

R₄ and R₅ are independently hydrogen, alkyl, alkenyl, alkynyl, cycloalkyl, cycloalkenyl, aryl, aralkyl, aralkenyl, aralkynyl, heteroaryl, heteroarylalkenyl, heteroarylalkynyl, optionally substituted, to form a cyclical acetal and ketal.

Formula I, IA, and IB compounds are useful as inhibitors of chemotaxis. Formula I, IA, and IB compounds can also be used as controls in chemotaxis assays.

### Preparation of the Compounds

In many cases, commercially available starting materials can be used for the synthesis of compounds of this invention. When not available commercially, useful starting materials can either be obtained from stepwise modification of commercially-available compounds and derivatives, or they may be synthesized from simpler precursors using literature methods known in the art. Other appropriate intermediates can be purchased from commercial sources and used as starting materials for compounds of the present invention, or can be synthesized as described in the chemical literature.

General approaches for preparations of some compounds of Formula I are described in Scheme 1 below. Those having skill in the art will recognize that the starting materials can be varied and additional steps employed to produce compounds encompassed by the present invention, as shown in the above schemes and as demonstrated by the examples which follow. In some cases, protection of certain reactive functionalities may be necessary to achieve some of the above transformations. In general, the need for such protecting groups as well as the conditions necessary to attach and remove such groups will be apparent to those skilled in the art.

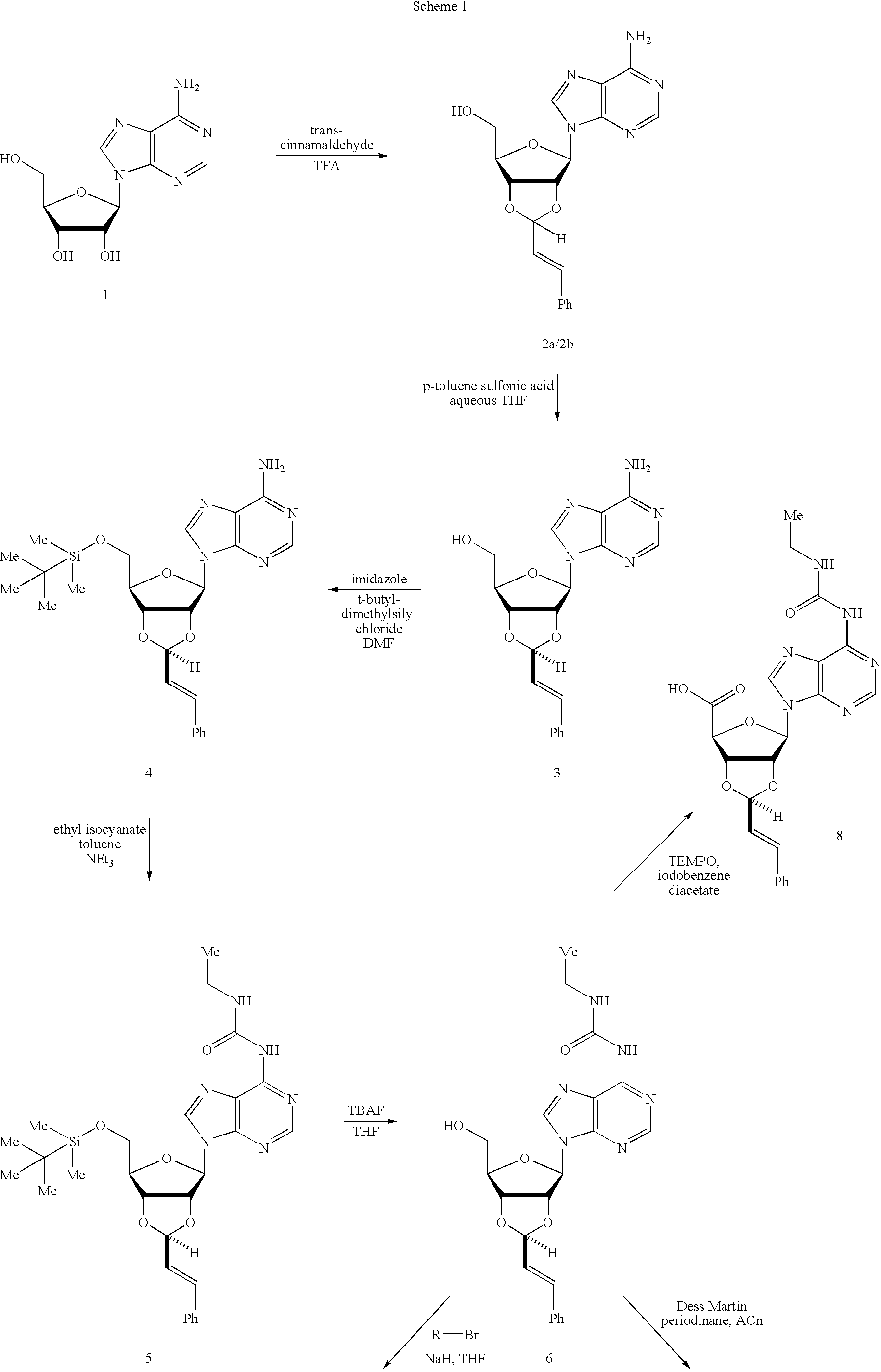

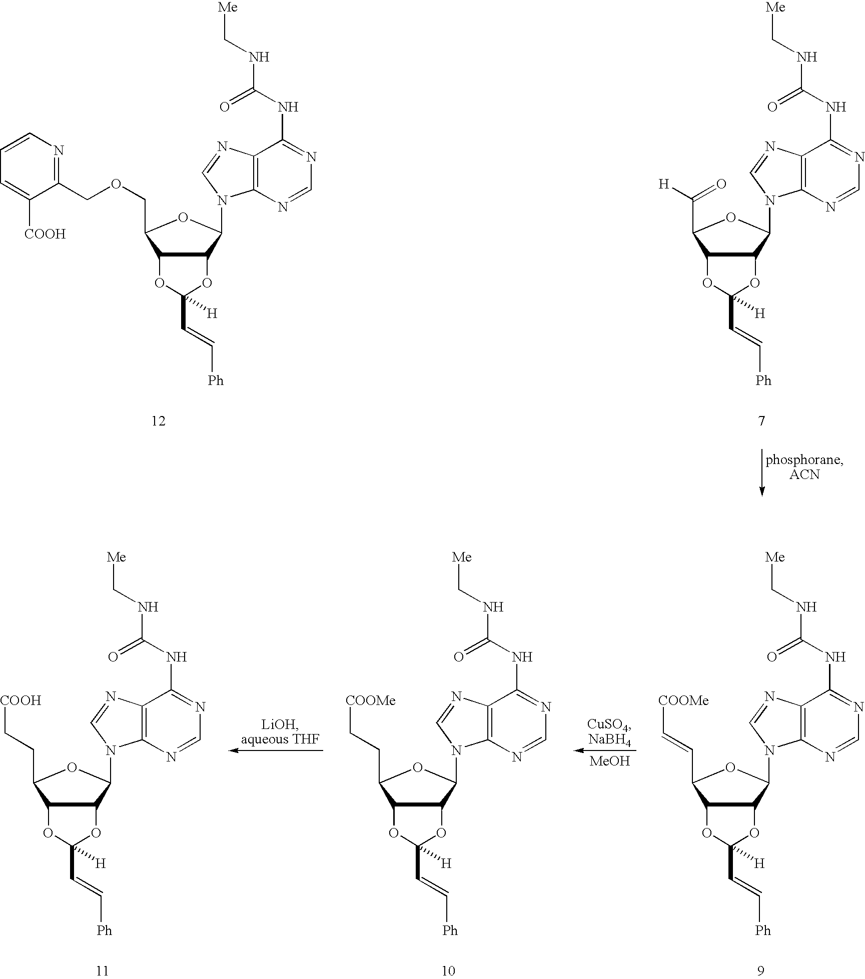

The present invention provides a method for preventing or treating inflammation by administering to a subject a compound of Formula I, IA, or IB.

In one embodiment, the Formula I compound is wherein R_{c}=H, G=O, R_{d′}=H, R_{d}=ethyl, cyclopentyl or phenyl, Q₂=CH₂OCH₂ or CH₂NHCH₂, A=formula II where R₆=H, r=0, p=1, X₁—X₆=C, M=H, R₄=H, R₅=benzyl or phenyl or styryl or phenylacetylynyl or butyl. Such compounds include Compound 12 and the following compounds:

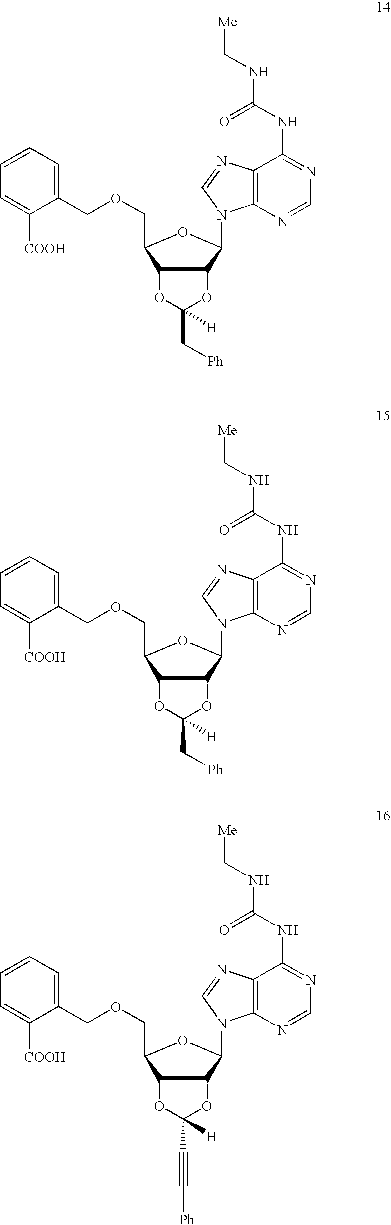

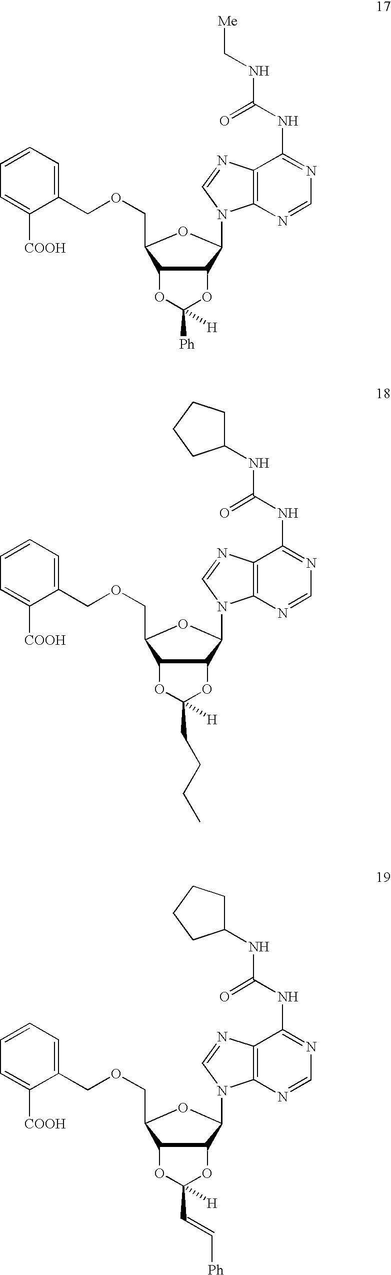

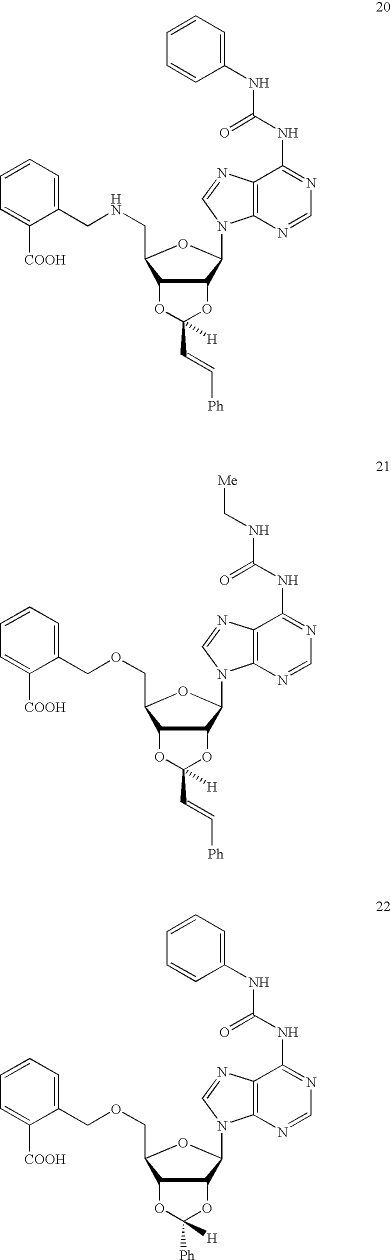

In another embodiment, the Formula IB compound is wherein R_{d′}=C₁₋₈ alkyl, C₃₋₇ cycloalkyl, aryl, or aralkyl, (e.g. ethyl, cyclopentyl or phenyl), Q₂=C₁₋₈ alkyl (e.g. CH₂CH₂), A=COOH, R₄=H, and R₅=aryl, aralkyl, aralkenyl (such as benzyl, styryl or phenyl). Such compounds include Compound 11 and the following compounds:

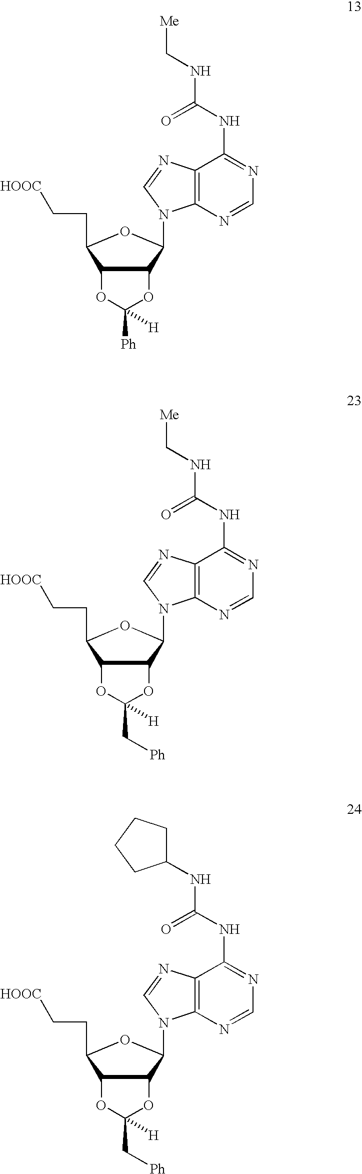

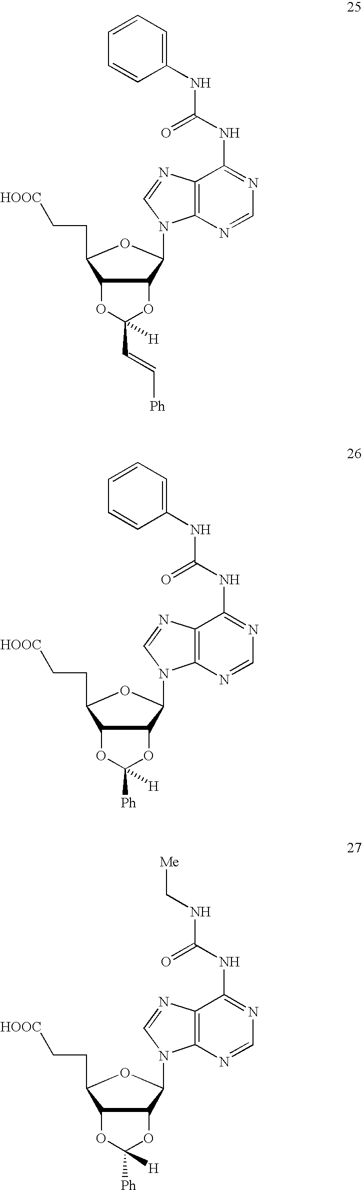

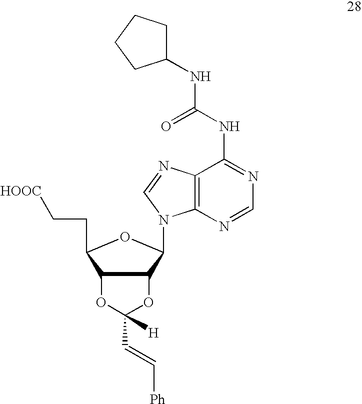

In yet another embodiment, the Formula IA compound is wherein R_{c}=H, G=O, R_{d}=H, R_{d′}=C₁₋₈ alkyl (e.g. ethyl), Q₂=C₁₋₈ alkyl (e.g. CH₂ or CHCH) or —C(O)—, A=CH₂COOH, OH, H, or COOCH₃, R₄=H, and R₅=styryl. Such Compounds include Compounds 6-10.

### Pharmaceutical Formulations

The present invention additionally provides pharmaceutical formulations comprising compounds of Formula I and a pharmaceutically acceptable carrier. Pharmaceutically acceptable carriers can be selected by those skilled in the art using conventional criteria. Pharmaceutically acceptable carriers include, but are not limited to, saline solution, aqueous electrolyte solutions, isotonicy modifiers, water polyethers such as polyethylene glycol, polyvinyls such as polyvinyl alcohol and povidone, cellulose derivatives such as methylcellulose and hydroxypropyl methylcellulose, polymers of acrylic acid such as carboxypolymethylene gel, polysaccharides such as dextrans, and glycosaminoglycans such as sodium hyaluronate and salts such as sodium chloride and potassium chloride.

The pharmaceutical formulation of the present invention provides an aqueous solution comprising water, suitable ionic or non-ionic tonicity modifiers, suitable buffering agents, and a compound of Formula I. In one embodiment, the compound is at 0.005 to 3% w/v, and the aqueous solution has a tonicity of 200-400 mOsm/kG and a pH of 4-9.

The pharmaceutical formulation can be sterilized by filtering the formulation through a sterilizing grade filter, preferably of a 0.22-micron nominal pore size. The pharmaceutical formulation can also be sterilized by terminal sterilization using one or more sterilization techniques including but not limited to a thermal process, such as an autoclaving process, or a radiation sterilization process, or using pulsed light to produce a sterile formulation. In one embodiment, the pharmaceutical formulation is a concentrated solution of the active ingredient; the formulation can be serially diluted using appropriate acceptable sterile diluents prior to intravenous administration.

In one embodiment, the tonicity modifier is ionic such as NaCl, for example, in the amount of 0.5-0.9% w/v, preferably 0.6-0.9% w/v.

In another embodiment, the tonicity modifier is non-ionic, such as mannitol, dextrose, in the amount of at least 2%, or at least 2.5%, or at least 3%, and no more than 7.5%; for example, in the range of 3-5%, preferably 3.5-5%, and more preferably 4.2-5% w/v.

Those skilled in the art will recognize various synthetic methodologies which may be employed to prepare non-toxic pharmaceutically acceptable salts and prodrugs of the compounds.

### Methods of Administration

The compounds of the invention can be administered orally, topically, parenterally, by inhalation or spray or rectally in dosage unit formulations containing conventional non-toxic pharmaceutically acceptable carriers, adjuvants and vehicles. Oral administration in the form of a pill, capsule, elixir, syrup, lozenge, troche, or the like is particularly preferred. The term parenteral as used herein includes injections and the like, such as subcutaneous, intradermal, intravascular (e.g., intravenous), intramuscular, intrasternal, spinal, intrathecal, and like injection or infusion techniques, with subcutaneous, intramuscular and intravascular injections or infusions being preferred. One or more compounds of the invention can be present in association with one or more non-toxic pharmaceutically acceptable carriers and/or diluents and/or adjuvants and if desired other active ingredients. The pharmaceutical compositions containing compounds of the invention can be in a form suitable for oral use, for example, as tablets, troches, lozenges, aqueous or oily suspensions, dispersible powders or granules, emulsion, hard or soft capsules, or syrups or elixirs (see Thurkauf et al, U.S. Pat. No. 6,884,815).

Compositions intended for oral use can be prepared according to any method known to the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or acetyl alcohol. Sweetening agents such as those set forth above, and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions can also contain sweetening and flavoring agents.

Syrups and elixirs can be formulated with sweetening agents, for example glycerol, propylene glycol, sorbitol or sucrose. Such formulations can also contain a demulcent, a preservative, a flavoring agent, and a coloring agent.

The pharmaceutical compositions can be in the form of a sterile injectable aqueous or oleaginous suspension. This suspension can be formulated according to the known art using those suitable dispersing or wetting agents and suspending agents which have been mentioned above. The sterile injectable preparation can also be sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The compounds of the invention can also be administered in the form of suppositories e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

Compounds of the invention can be administered parenterally, preferably in a sterile non-toxic, pyrogen-free medium. The drug, depending on the vehicle and concentration used, can either be suspended or dissolved in the vehicle. Advantageously, adjuvants such as local anesthetics, preservatives and buffering agents can be dissolved in the vehicle.

Dosage levels about 0.01-140 mg per kg of body weight per day are useful in the treatment or preventions of conditions involving an inflammatory response (about 0.5 mg to about 7 g per patient per day). Preferred dosage levels are about 0.05-25, or 0.1-10 mg/kg body weight per day. The amount of active ingredient that can be combined with the carrier materials to produce a single dosage form will vary depending upon the host treated and the particular mode of administration. Dosage unit forms will generally contain between from about 1 mg to about 500 mg of an active ingredient.

Frequency of dosage can also vary depending on the compound used and the particular disease treated. However, for treatment of most disorders, a dosage regimen of 4 times daily, three times daily, or less is preferred, with a dosage regimen of once daily or 2 times daily being particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner.

Preferred compounds of the invention have favorable pharmacological properties. Such properties include, but are not limited to bioavailability (e.g., oral bioavailibilty, preferably high enough to permit oral administration of doses of less than 2 grams, preferably of less than or equal to one gram), low toxicity, low serum protein binding and desirable in vitro and in vivo half-life.

Assays can be used to predict these desirable pharmacological properties. Assays used to predict bioavailability include transport across human intestinal cell monolayers, including Caco-2 cell monolayers. Toxicity to cultured hepatocycles can be used to predict compound toxicity.

In one embodiment, the compound of Formula I is topically administered in a form selected from the group consisting of a solution, a gel, a suspension, a cream, and an ointment containing the compound in a physiologically compatible vehicle.

In another embodiment, the compound of Formula I is systemically administered in a form selected from the group consisting of an aerosol suspension of respirable particles, a liquid or liquid suspension for administration as nose drops or nasal spray, a nebulized liquid for administration to oral or nasopharyngeal airways, an oral form, an injectable form, a suppository form, and a transdermal patch or a transdermal pad.

In yet embodiment, the compound of Formula I is administered by direct intra-operative instilling a form selected from the group consisting of a gel, a cream, and a liquid suspension form.

The invention is illustrated further by the following example that is not to be construed as limiting the invention in scope to the specific procedures described in it.

### EXAMPLES

### Example 1

### Preparation of cis-[6-(6-amino-purin-9-yl)-2-styryl-tetrahydro-furo[3,4-d][1,3]dioxol-4-yl]-methanol (2a) and trans-[6-(6-amino-purin-9-yl)-2-styryl-tetrahydro-furo[3,4-d][1,3]dioxol-4-yl]-methanol (Compound 2b)

A 3 L flask equipped with a mechanical stirrer, addition funnel, internal temperature monitor and nitrogen inlet was flushed with nitrogen and charged with 90 g of (−)-adenosine (1) and 0.339 L of trans-cinnamaldehyde. After cooling (acetone/wet ice bath) to −5⁰ C., 0.403 L of trifluoroacetic acid was added keeping the temperature between −5⁰ C. and +5⁰ C. The reaction was stirred at 0⁰ C. until 80% conversion is achieved (approximately 2 hours, as measured by HPLC). The reaction was then diluted with 1.1 L of iso-propyl acetate maintaining a reaction temperature of <5⁰ C. The reaction was then quenched with 0.810 L of 5 N sodium hydroxide maintaining a reaction temperature of 20⁰ C. to 25⁰ C. During this quench, the product crystallized and two layers were formed. After the addition was complete, agitation was stopped and the layers were allowed to separate. The product settled into the bottom of the top organic phase. The lower aqueous phase was decanted and agitation was continued. The product was isolated by filtration and washed with 3×0.450 L of iso-propyl acetate. The resulting solid was dried on a filter and then transferred to an oven and dried to a constant weight under vacuum at 50⁰ C. Approximately 85-90 g of a mixture of cis-[6-(6-amino-purin-9-yl)-2-styryl-tetrahydro-furo[3,4-d][1,3]dioxol-4-yl]-methanol (1a) and trans-[6-(6-amino-purin-9-yl)-2-styryl-tetrahydro-furo[3,4-d][1,3]dioxol-4-yl]-methanol (1b) was obtained.

### Example 2

### Preparation of trans-[6-(6-amino-purin-9-yl)-2-styryl-tetrahydro-furo[3,4-d][1,3]dioxol-4-yl]-methanol (Compound 3)

A 1 L flask equipped with a mechanical stirrer, addition funnel, internal temperature monitor and nitrogen inlet was flushed with nitrogen and charged with 50 g of a 1.5:1 mixture of trans:cis-[6-(6-amino-purin-9-yl)-2-styryl-tetrahydro-furo[3,4-d][1,3]dioxol-4-yl]-methanol (2a/2b), 18.6 g of p-toluene sulfonic acid, 0.2 L of tetrahydrofuran and 0.050 L of water. The reaction was warmed to 50⁰ C. and stirred until the HPLC area % ratio of trans-[6-(6-amino-purin-9-yl)-2-styryl-tetrahydro-furo[3,4-d][1,3]dioxol-4-yl]-methanol to cis-[6-(6-amino-purin-9-yl)-2-styryl-tetrahydro-furo[3,4-d][1,3]dioxol-4-yl]-methanol was >99:1.0. The reaction was then quenched with 0.150 L of 2 sodium hydroxide and stirred for 10 minutes. Agitation was stopped and the phases allowed to separate. The lower aqueous phase was decanted and agitation was continued. The reaction was then diluted with 0.200 L of iso-propyl acetate and allowed to cool to 20⁰ C. The product was isolated by filtration and washed with 0.2 L of iso-propyl acetate. The resulting paste was dried on a filter until it was tractable enough to manipulate. The cake cracked and separated and required pressing to invoke further solvent removal. The resulting solid was dried on a filter and then transferred to an oven and dried to a constant weight under vacuum at 50⁰ C. Approximately 19 g of trans-[6-(6-amino-purin-9-yl)-2-styryl-tetrahydro-furo[3,4-d][1,3]dioxol-4-yl]-methanol (3) was obtained.

### Example 3

### Preparation of trans-9-[6-(tert-butyl-dimethyl-silanyloxymethyl)-2-styryl-tetrahydro-furo[3,4-d][1,3]dioxol-4-yl]-9H-purin-6-ylamine (Compound 4)

A 3 L flask equipped with a mechanical stirrer, addition funnel, internal temperature monitor, nitrogen inlet and vacuum line was flushed with nitrogen and charged with 100 g of trans-[6-(6-amino-purin-9-yl)-2-styryl-tetrahydro-furo[3,4-d][1,3]dioxol-4-yl]-methanol (3) and 2 L of iso-propyl acetate. The reaction was warmed to reflux and 0.600 L of distillate was collected. The reaction was then charged with 1.0 L of N,N-dimethyl formamide and distillate was collected until a pot temperature of 100⁰ C was reached at a pressure of 200 torr. The reaction was then cooled to 20⁰ C. The reaction was then charged with 0.0223 kg of imidazole and 0.0474 kg of tert-butyl dimethylsilyl chloride. After stirring for two hours the reaction was tested for completeness by HPLC. The reaction was then quenched with 1.4 L of a 2.5:1 mixture of water/2-propanol keeping the internal temperature between 15-20⁰ C. After stirring for 1 hour the product was isolated by filtration and washed with 1.2 L of 2-propanol. The solid was dried to constant weight in a vacuum oven at 50⁰ C. Approximately 105 g of trans-9-[6-(tert-butyl-dimethyl-silanyloxymethyl)-2-styryl-tetrahydro-furo[3,4-d][1,3]dioxol-4-yl]-9H-purin-6-ylamine (4) was obtained.

### Example 4

### Preparation of trans-1-{9-[6-(tert-butyl-dimethyl-silanyloxymethyl)-2-styryl-tetrahydro-furo[3,4-d][1,3]dioxol-4-yl]-9H-purin-6-yl}-3-ethyl-urea (Compound 5)

A 2 L flask equipped with a mechanical stirrer and reflux condenser was flushed with nitrogen and charged with 0.088 kg of trans-9-[6-(tert-butyl-dimethyl-silanyloxymethyl)-2-styryl-tetrahydro-furo[3,4-d][1,3]dioxol-4-yl]-9H-purin-6-ylamine (4), 0.352 L of toluene, 0.0454 L of ethyl isocyanate and 0.0247 L of triethyl amine. The reaction was warmed to 60⁰ C. The reaction was run for 40 hours and then assayed for completeness. The reaction was diluted with 0.300 L of toluene and 0.380 L of distillate was collected. The reaction was cooled to 100⁰ C. and 0.881 L of heptane was added slowly. The resulting temperature was 55⁰ C. The product crystallized upon cooling to room temperature. It was then isolated by filtration and washed with 2×0.2 L of heptane. The solid was dried to a constant weight in a vacuum oven at <50⁰ C. Approximately 0.040 Kg of the title compound (5) was obtained.

### Example 5

### Preparation of trans-1-ethyl-3-[9-(6-hydroxymethyl-2-styryl-tetrahydro-furo[3,4-d][1,3]dioxol-4-yl)-9H-purin-6-yl]-urea (Compound 6)

A 2 L flask equipped with a mechanical stirrer was flushed with nitrogen and charged with 0.094 kg of trans-1-{9-[6-(tert-butyl-dimethyl-silanyloxymethyl)-2-styryl-tetrahydro-furo[3,4-d][1,3]dioxol-4-yl]-9H-purin-6-yl}-3-ethyl-urea (5) and 0.0235 L of THF. Tetrabutyl ammonium fluoride (TBAF; 0.249 L of a 1 M solution in THF solution) was then added and the reaction stirred until complete by HPLC. A solution of 0.047 L of 1 N hydrochloric acid and 0.191 L of water was then added. The product crystallized upon stirring at 20⁰ C. The solid was then isolated by filtration and washed with 2×0.500 L of 2-propanol. The solid was dried to a constant weight in a vacuum oven at <50⁰ C. Approximately 0.062 kg of the title compound (6) was obtained.

### Example 6

### Preparation of trans-3-{6-[6-(3-ethyl-ureido)-purin-9-yl]-2-styryl-tetrahydro-furo[3,4-d][1,3]dioxol-4-yl}-acrylic acid methyl ester (Compound 2)

Trans-1-ethyl-3-[9-(6-hydroxymethyl-2-styryl-tetrahydro-furo[3,4-d][1,3]dioxol-4-yl)-9H-purin-6-yl]-urea (6, 5.0 g, 11 mmol) was suspended in dry acetonitrile (50 mL) and Dess-Martin periodinane (6.7 g, 16 mmol) was added. The suspension was stirred 2 h, after which time proton NMR of an aliquot showed complete conversion to the aldehyde (2). (Methoxycarbonylmethylene)triphenylphosphorane (3.9 g, 12 mmol) was added and stirring was continued overnight. The reaction mixture was then diluted with ethyl acetate (300 mL), washed with saturated sodium bicarbonate/thiosulfate solution (100 mL), dried with sodium sulfate and filtered. The filtrate was evaporated and the solid was dissolved in hot isopropyl alcohol (50 mL). It was allowed to cool, then heptane was added and it was stirred overnight. The resulting precipitate was washed with heptane and dried under vacuum, affording the desired product (9, 2.9 g, 71%). ¹H-NMR (300 MHz, d₆ DMSO) δ 1.15 (t, 3H, J=7 Hz), 3.21 (q, 2H, J=7 Hz), 3.59 (s, 3H), 4.98 (m, 1H), 5.28 (t, 1H, J=6 Hz), 5.51 (dd, 1H, J=6 Hz, <2 Hz), 5.70 (d, 1H, J=16 Hz), 5.90 (d, 1H, J=6 Hz), 6.30 (dd, 1H, J=6 Hz, 16 Hz), 6.45 (d, 1H, J<2 Hz), 6.95 (d, 1H, J=15 Hz), 7.35 (m, 3H), 7.45 (d, 2H, J=7 Hz), 8.50 (s, 1H), 8.60 (s, 1H), 9.30 (t, 1H, J=6 Hz), 9.60 (s, 1H).

### Example 7

### Preparation of trans-3-{6-[6-(3-ethyl-ureido)-purin-9-yl]-2-styryl-tetrahydro-furo[3,4-d][1,3]dioxol-4-yl}-propionic acid methyl ester (Compound 10)

Trans-3-{6-[6-(3-ethyl-ureido)-purin-9-yl]-2-styryl-tetrahydro-furo[3,4-d][1,3]dioxol-4-yl}-acrylic acid methyl ester (2, 250 mg, 0.5 mmol) was dissolved in dry methanol (3 mL). Copper (II) sulfate (90 mg, 0.5 mmol) was added followed by sodium tetrahydroborate (90 mg, 2.5 mmol) and the reaction was stirred 48 h. The reaction was diluted with water, filtered, and concentrated in vacuo. The residue was dissolved in ethyl acetate and precipitated with heptane. The precipitate was dissolved in dichloromethane and was chromatographed on silica gel with dichloromethane-methanol (95:5) as eluent, affording the title compound (10, 125 mg, 50%). ¹H-NMR (300 MHz, d₆DMSO) δ 1.15 (t, 3H, J=7 Hz), 1.90 (m, 2H), 2.19 (m, 2H), 3.21 (q, 2H, J=7 Hz), 3.55 (s, 3H), 4.20 (m, 1H), 4.98 (dd, 1H, J=4 Hz, 6 Hz), 5.45 (dd, 1H, J=3 Hz, 7 Hz), 5.85 (d, 1H, J=6 Hz), 6.25 (d, 1H, J=3 Hz), 6.27 (dd, 1H, J=6 Hz, 16 Hz), 6.90 (d, 1H, J=16 Hz), 7.35 (m, 3H), 7.50 (d, 2H, J=7 Hz), 8.56 (s, 1H), 8.57 (s, 1H), 9.30 (t, 1H, J=5 Hz), 9.60 (s, 1H).

### Example 8

### Preparation of trans-3-{6-[6-(3-ethyl-ureido)-purin-9-yl]-2-styryl-tetrahydro-furo[3,4-d][1,3]dioxol-4-yl}-propionic acid (Compound 11)

Trans-3-{6-[6-(3-ethyl-ureido)-purin-9-yl]-2-styryl-tetrahydro-furo[3,4-d][1,3]dioxol-4-yl}-propionic acid methyl ester (10, 5.0 g, 10 mmol) was dissolved in tetrahydrofuran (300 mL). Water (100 mL) was added, followed by lithium hydroxide (1.0 g, 25 mmol). The solution was allowed to stir 16 h at room temperature. It was acidified to pH 5 with acetic acid, concentrated in vacuo, then extracted with chloroform (300 mL). The organic extract was evaporated, redissolved in ethyl acetate and precipitated with heptane to afford the final product (11, 3.9 g, 80%). ¹H-NMR (300 MHz, d₆DMSO) δ 1.14 (t 3H, J=7 Hz), 1.90 (m, 2H), 2.19 (m, 2H), 3.26 (q, 2H, J=6 Hz), 4.17 (m, 1H), 4.93 (t, 1H, J=6 Hz), 5.43 (dd, 1H, J=3 Hz, 7 Hz), 5.84 (d 1H, J=6 Hz), 6.24 (d, 1H, J=3 Hz), 6.28 (dd, 1H, J=7 Hz, 13 Hz), 6.90 (d, 1H, J=16 Hz), 7.35 (m, 3H), 7.51 (d, 2H, J=7 Hz), 8.56 (s, 1H), 8.61 (s, 1H), 9.30 (t, 1H, J=6 Hz).

### Example 9

### Preparation of trans-1-ethyl-3-[9-(6-carboxy-2-styryl-tetrahydro-furo[3,4-d][1,3]dioxol-4-yl)-9H-purin-6-yl]-urea (Compound 8)

Trans-1-ethyl-3-[9-(6-hydroxymethyl-2-styryl-tetrahydro-furo[3,4-d][1,3]dioxol-4-yl)-9H-purin-6-yl]-urea (6, 10 g, 22 mmol) was dissolved in a degassed mixture of acetonitrile (60 mL) and water (60 mL). To this was added iodobenzene diacetate (16 g, 49 mmol), followed by 2,2,6,6-tetramethyl-1-piperidinyloxy, free radical (TEMPO, 0.7 g, 4 mmol) and the reaction was stirred for 3 hours at ambient temperature. The solvents were removed and the residual oil was converted to a solid by trituration with ethyl ether. Filtration and drying afforded the title compound (8, 8.56 g, 83% yield). ¹H-NMR (300 MHz, CDCl₃) δ 1.30 (t, 3H), 3.46 (m, 2H), 5.03 (s, 1H), 5.36 (m, 1H), 5.56 (m, 1H), 5.94 (d, 1H), 6.15 (dd, 1H), 6.51 (d, 1H), 6.87 (d, 1H), 7.35 (m, 5H), 8.48 (s, 1H), 9.1 (s, 1H), 9.96 (t, 1H), 10.24 (s, 1H).

### Example 10

### Preparation of trans-2-{6-[6-(3-ethyl-ureido)-purin-9-yl]-2-styryl-tetrahydro-furo-[3,4-d][1,3]dioxol-4yl methoxymethyl}-nicotinic acid (Compound 12)

Trans-1-ethyl-3-[9-(6-hydroxymethyl-2-styryl-tetrahydro-furo[3,4-d][1,3]dioxol-4-yl)-9H-purin-6-yl]-urea (6, 250 mg, 0.55 mmol) was dissolved in dry N,N-dimethylformamide (2 mL) and 3-carboxyethyl-2-bromomethylpyridine hydrochloride (155 mg, 0.55 mmol) added. 60% sodium hydride (133 mg, 3.3 mmol) was added and the reaction was stirred at ambient temperature under nitrogen. After overnight stirring, the reaction was about 50% complete, so a further portion of sodium hydride (50 mg, 1.25 mmol) was added. After one more hour stirring, the reaction was quenched with acetic acid. The title compound (12, obtained directly in the reaction via in situ hydrolysis of the ester) was isolated via preparative HPLC using a gradient from 0.025 M ammonium acetate (pH 6) to acetonitrile. Yield 155 mg (48%): ¹H-NMR (300 MHz, d₆ DMSO) δ 1.06 (t, 3H), 3.3 (q, 2H), 3.68 (m, 2H), 4.51 (m, 1H), 4.82 (q, 2H), 5.09 (m, 1H), 5.57 (m, 1H), 6.12 (s, 1H), 6.19 (s, 1H), 7.41 (m, 4H), 8.55 (s, 1H), 8.72 (s, 1H), 9.18 (t, 1H).

### Example 11

### Preparation of Compound 13

Compound 13 was prepared in an identical manner to 11, substituting benzaldehyde for cinnamaldehyde in the first step, followed by elaboration to 13 using the methods of examples 2-8. Compound 13: ¹H-NMR (300 MHz, d₆DMSO) δ 1.05 (t, 3H), 1.94 (m, 2H), 2.13 (m, 2H), 3.4 (q, 2H), 4.12 (m, 1H), 5.01 (m, 1H), 5.59 (m, 1H), 6.21 (s, 1H), 6.3 (d, 1H), 7.42 (m, 5H), 8.59 (s, 1H), 8.62 (s, 1H), 9.26 (t, 1H), 9.58 (s, 1H).

### Example 12

### Neutrophil Isolation

Neutrophils were isolated from peripheral blood donated by healthy volunteers. Twenty milliliters of whole blood containing ACD (Ascorbic Acid/Citrate/Dextrose) as an anticoagulant was layered over twenty milliliters Ficoll-Pacque Plus (Stem Cell Technologies, Vancouver, BC) and centrifuged for 45 minutes at 1500 rpm. Following centrifugation, the red blood cell layer (also containing the granulocytes) at the bottom of the tube was retained in the centrifugation tube while the top layers were removed. The red blood cell and granulocytes were combined with an equal volume of 3% Dextran 500 (Amersham Biosciences, Uppsala, Sweden) and mixed gently. The red blood cells were allowed to sediment for 30 minutes. The top layer containing the granulocytes was removed to a new centrifuge tube. The remaining red blood cells were lysed through hypotonic shock. This was accomplished through exposure of the cells to an ice-cold 0.2% NaCl (Sigma-Aldrich, St. Louis, Mo.) solution for 30 seconds, followed by addition of an ice-cold 1.6% NaCl solution to bring the total NaCl concentration back to 0.9%. The granulocytes were recovered through centrifugation for ten minutes at 1400 rpm. The cells were resuspended in Hank's Balanced Salt Solution (HBSS, Invitrogen, Grand Island, N.Y.), and counted on a hemacytometer. The volume was adjusted to bring the cell density to 1.25 million cells per milliliter. Cell viability was determined through trypan blue exclusion and was greater than 95%. Samples were spotted on microscope slides and subsequently stained with Diff-Quik (Dade Behring Inc., Newark, Del.). Microscopic analysis following staining identified greater than 95% of cells as neutrophils.

### Example 13

### Chemoattractants

Chemoattractant compounds (e.g. fMLP) are prepared as 20 mM stock solutions and dissolved in either DMSO or water. A Boyden type chemotaxis chamber is used to test the neutrophil chemotaxis response. Chemoattractant solutions are diluted in HBSS (concentrations ranging from 1 mM to 0.1 nM) and placed in the bottom chambers of the chemotaxis apparatus. A 3 μm pore size, PVP free polycarbonate filter membrane is placed over the lower chambers. Isolated neutrophils suspended in HBSS are added to the upper chamber (500,000 to 2,500,000 cells/mL) of the chemotaxis apparatus. The chemotaxis apparatus is placed in an incubator at 37⁰ C. and incubated for one to three hours. The apparatus is disassembled and the filter membrane with the migrated cells stained with Diff Quik (Dade Behring Inc, Newark, Del.). The number of migrated cells is determined by microscopy and counting the number of cells per high powered field.

A significant increase in the number of migrated neutrophils is observed in the wells containing an effective concentration of a chemoattractant.

### Example 14

### Inhibition of Neutrophil Chemotaxis

Inhibitors of neutrophil chemotaxis are identified using a Boyden type chemotaxis chamber. Chemotaxis inhibitor compounds are prepared as 20 millimolar stock solutions in either DMSO or water. Stock solutions of chemotaxis inhibitors, such as Compound 11, trans-3-{6-[6-(3-ethyl-ureido)-purin-9-yl]-2-styryl-tetrahydro-furo[3,4-d][1,3]dioxol-4-yl}-propionic acid, are diluted (concentrations ranging from μM to 0.1 pM) in HBSS containing a chemoattractant and placed in the lower chamber of the apparatus. A 3 μm pore size, non PVP coated polycarbonate filter membrane is placed over the lower chamber. Isolated neutrophils suspended in HBSS (containing the matching concentration of the chemotaxis inhibitor in the lower chamber), are added to the upper chamber (500,000 to 2,500,000 cells/mL) and incubated for one to three hours at 37⁰ C. The apparatus is disassembled and the filter membrane with the migrated cells stained with Diff Quik (Dade Behring Inc, Newark, Del.). The number of migrated cells is determined by microscopy and counting the number of cells per high powered field.

A significant decrease is observed in the number of migrated neutrophils in the wells containing effective concentrations of inhibitors of neutrophil chemotaxis, such as Compound 11.

### Example 15

### Treatment of Acute Pulmonary Inflammation in Rat by INS55506

The administration of lipopolysaccharides (LPS) from gram-negative bacteria cell walls to the lungs of rats results in a rapid increase in the total number of leukocytes in the lumen of the lungs. The increase of neutrophils in the lung in the LPS model represents the conditions present a patients with chronic respiratory diseases such as cystic fibrosis, chronic bronchitis, emphysema, and certain subtypes of asthma. The LPS model of acute pulmonary inflammation is a well-established model for testing compounds having potential activities in treating inflammatory airway diseases.

To demonstrate the ability to reduce the inflammatory response in vivo, Compound 11 was administered to fasted rats prior to and following pulmonary administration of the aerosolized LPS. Compound 11 (35 mg/kg) or vehicle (0.5% aqueous methylcellulose) was orally administered to groups of rats 15 minutes prior to pulmonary aerosolized LPS (˜2 micrograms/rat) or placebo (air) exposure (20 minute exposure) and 2.5 and 5 hours following the initial oral administration. Eight hours after pulmonary administration of aerosolized LPS, the lungs were lavaged to obtain bronchoalveolar lavage fluid, containing accumulated leukocytes.

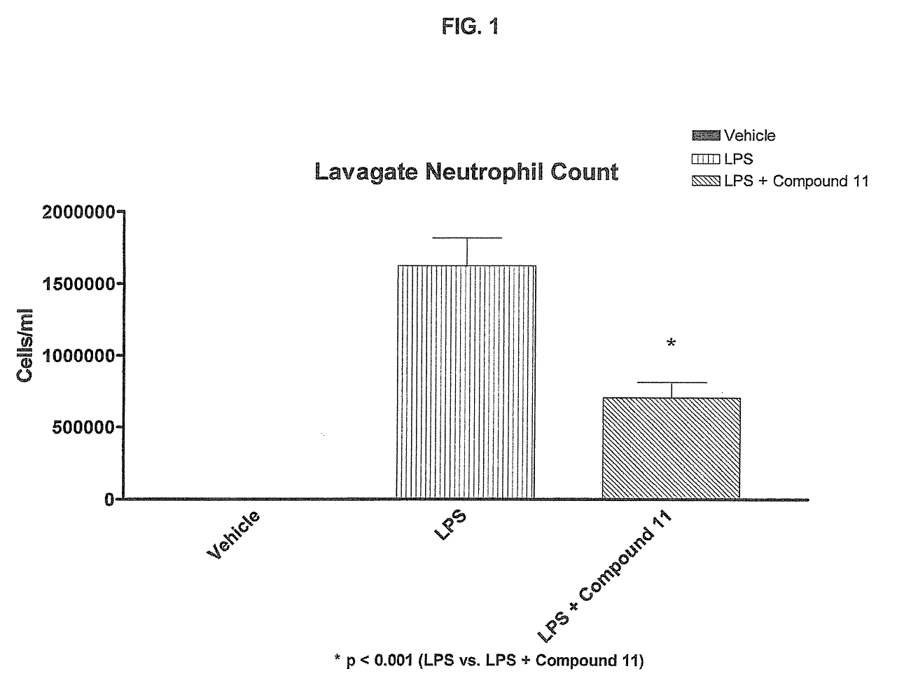

A significant decrease (56.6%, p<0.001) was observed in the number of neutrophils recovered in the lavage fluid from groups of rats receiving the Compound 11 and LPS when compared with the lavage fluid from groups of rats receiving vehicle and LPS. Data from this experiment is shown in FIG. 1. The reduction of neutrophils in the LPS model is a clear indication of reduced inflammation.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed:

1. A method of preventing or treating diseases or conditions associated with inflammation in a mammal comprising:
administering to a mammal in need thereof an effective amount of a compound of Formula IA, or a pharmaceutically acceptable salt, tautomer, hydrate, or solvate thereof, wherein said amount is effective to inhibit inflammation, 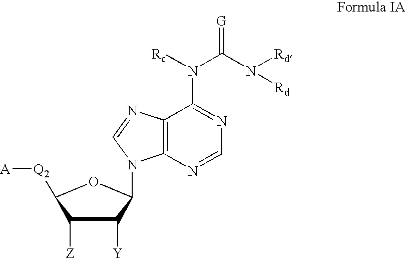
wherein;
Q₂=C₁₋₈ alkyl, C₂₋₈ alkenyl, C₂₋₈ alkynyl, optionally containing one or more heteroatoms, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ heterocycle, —(CO)—, or absent;
A is H, —OR, —SR, —C₁₋₃ alkyl, C₂₋₃ alkenyl, or C₂₋₃ alkynyl, and each hydrogen of said alkyl or alkenyl is optionally substituted by 0 to 2 fluorine groups, 0 to 1 methyl group, 0 to 2 —[(CO)OR] groups, or 0 to 1 —(OR) group;
R_{c}=H;
G=O;
R_{d} and R_{d′} are independently selected from the group consisting of: H, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, and C₂₋₆ heterocycle, where all rings or chains optionally bear one or more desired substituents; or
R_{d} and R_{d′} groups are taken together to form a ring of 4 to 7 members, with or without unsaturation and with or without heteroatoms in place of ring-carbon units;
Y=H, OH, or ORₐ;
Z=H, OH, or OR_{b}; with the proviso that Y and Z are not both H;
R is selected from the group consisting of: H, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, and C₂₋₆ heterocycle; where all rings or chains optionally bear one or more desired substituents;
Rₐ and R_{b} are residues which are linked directly to the 2′ and/or 3′ oxygens of the furanose via a carbon atom according to Formula III, or linked directly to the two 2′ and 3′ oxygens of the furanose via a common carbon atom according to Formula IV; 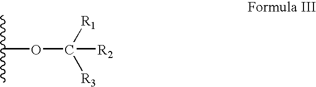
wherein 0 is the corresponding 2′ and/or 3′ oxygen of the furanose;
R₁, R₂, and R₃ are independently H, alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted, such that the moiety defined by Formula III is an ether; or
R₁ and R₂ are independently H, alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted; and R₃ is alkoxy, cycloalkoxy, aralkyloxy, aryloxy, substituted aralkyloxy, or substituted aryloxy, such that the moiety defined by Formula III is an acyclic acetal or ketal; or
R₁ and R₂ are taken together as oxygen or sulfur doubly bonded to C₁ and R₃ is alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted, such that the moiety defined by Formula III is an ester or thioester; or
R₁ and R₂ are taken together as oxygen or sulfur doubly bonded to C₁ and R₃ is amino or mono- or disubstituted amino, where the substituents are independently alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted, such that the moiety defined by Formula III is a carbamate or thiocarbamate; or
R₁ and R₂ are taken together as oxygen or sulfur doubly bonded to C₁ and R₃ is alkoxy, cycloalkoxy, aralkyloxy, aryloxy, substituted aralkyloxy, or substituted aryloxy, such that the moiety defined by Formula III is a carbonate or thiocarbonate; or
R₃ is not present and R₁ and R₂ are taken together as oxygen or sulfur doubly bonded to C and both the 2′ and 3′ oxygens of the furanose are directly bound to C to form a cyclical carbonate or thiocarbonate; 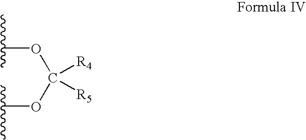
wherein 0 is the 2′ and 3′ oxygens of the furanose; and the 2′ and 3′ oxygens of the furanose are linked by a common carbon atom to form a cyclical acetal, cyclical ketal, or cyclical orthoester;
for the cyclical acetal and ketal, R₄ and R₅ are independently hydrogen, alkyl, alkenyl, alkynyl, cycloalkyl, cycloalkenyl, aryl, aralkyl, aralkenyl, aralkynyl, heteroaryl, heteroarylalkenyl, heteroarylalkynyl, optionally substituted; or
R₄ and R₅ are joined together to form a homocyclic or heterocyclic ring composed of 3 to 8 atoms, preferably 3 to 6 atoms;
for the cyclical orthoester, R₄ is hydrogen, alkyl, alkenyl, alkynyl, cycloalkyl, cycloalkenyl, aryl, aralkyl, aralkenyl, aralkynyl, heteroaryl, heteroarylalkenyl, heteroarylalkynyl, optionally substituted, and R₅ is alkyloxy, cycloalkyloxy, aralkyloxy, aryloxy, substituted aralkyloxy, or substituted aryloxy.

2. The method according to claim 1, wherein said compound is a compound of Formula IB, 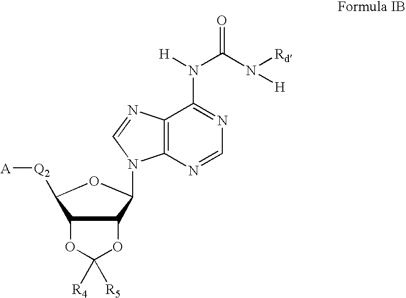
wherein Q₂=C₁₋₈ alkyl, C₂₋₈ alkenyl, C₂₋₈ alkynyl, optionally containing one or more heteroatoms, aralkyl, aralkenyl, aralkynyl, heteroaryl, heteroarylalkenyl, heteroarylalkynyl, C₂₋₆ heterocycle, or absent,
A is —OR, —SR, —COOR;
the first atom of the moiety A/Q₂ directly attached to the 4′ position is C;
R and R_{d′} are independently selected from the group consisting of: H, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ heterocycle; where all rings or chains optionally bear one or more desired substituents; and
R₄ and R₅ are independently hydrogen, alkyl, alkenyl, alkynyl, cycloalkyl, cycloalkenyl, aryl, aralkyl, aralkenyl, aralkynyl, heteroaryl, heteroarylalkenyl, heteroarylalkynyl, optionally substituted.

3. The method according to claim 2, wherein R_{d′}=C₁₋₈ alkyl, C₃₋₇ cycloalkyl, C₂₋₈ alkenyl, aryl, aralkyl, aralkenyl, Q₂=C₁₋₈ alkyl or absent, A=COOH, OH, or COOCH₃, R₄=H, and R₅=aryl, aralkyl, or aralkenyl.

4. The method according to claim 3, wherein said compound is Compound 11:

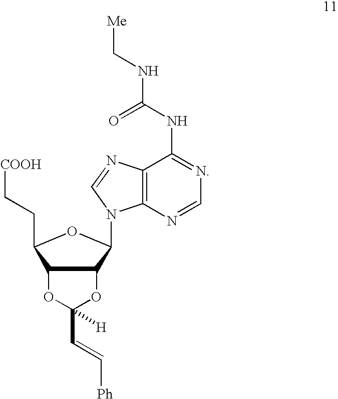

5. The method according to claim 3, wherein said compound is Compound 13, 23, 24, 25, 26, 27, or 28:

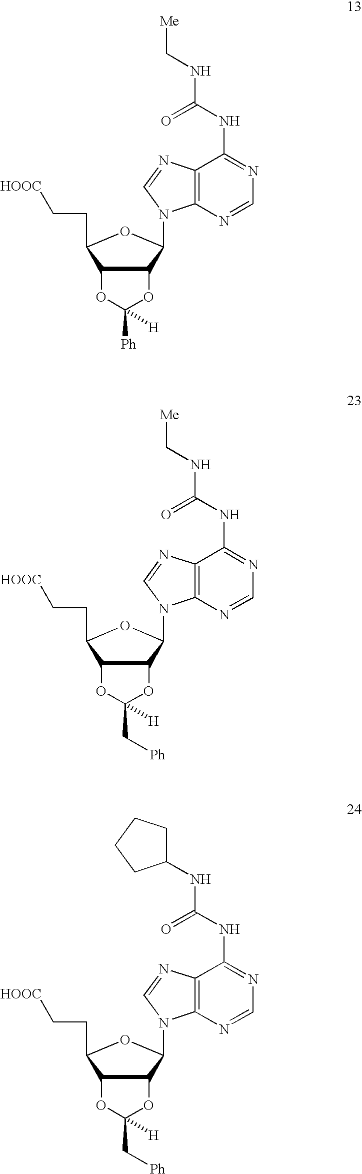

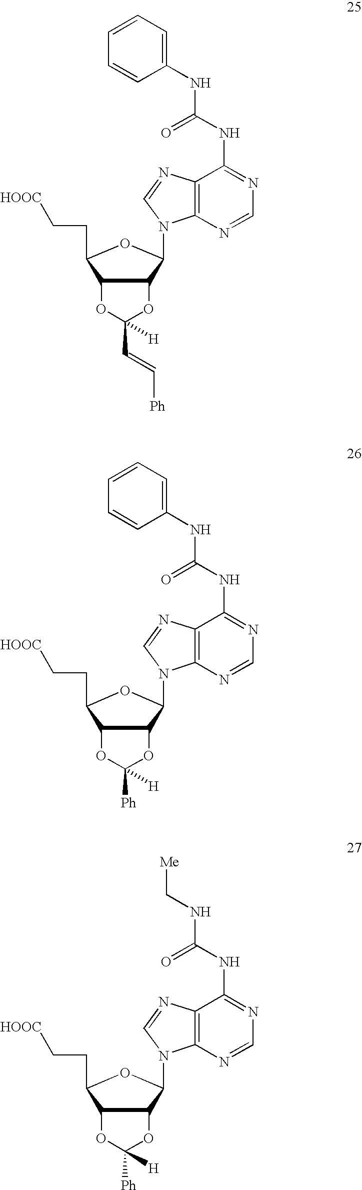

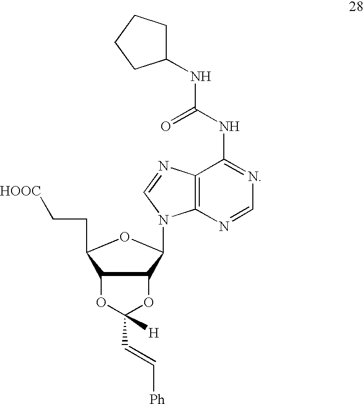

6. The method according to claim 1, wherein said diseases or conditions associated with inflammation are respiratory diseases associated with pulmonary inflammation.

7. The method according to claim 6, wherein said respiratory diseases are chronic obstructive pulmonary disease, cystic fibrosis, or asthma.

8. The method according to claim 7, wherein said chronic obstructive pulmonary disease is chronic bronchitis or emphysema.

9. The method according to claim 1, wherein said mammal is a human.

10. The method according to claim 1, wherein said administering is systemically administering a form selected from the group consisting of an aerosol suspension of respirable particles, a liquid or liquid suspension for administration as nose drops or nasal spray, a nebulized liquid for administration to oral or nasopharyngeal airways, an oral form, an injectable form, a suppository form, and a transdermal patch or a transdermal pad.

11. The method according to claim 1, wherein said administering is topical administering a form selected from the group consisting of a solution, a gel, a suspension, a cream, and an ointment containing the compound in a physiologically compatible vehicle.

12. A method for inhibiting chemotaxis of leukocytes, comprising contacting leukocytes with an effective amount of a compound of Formula IA, or a pharmaceutically acceptable salt, tautomer, hydrate, or solvate thereof, wherein said amount is effective to inhibit chemotaxis of the leukocytes. 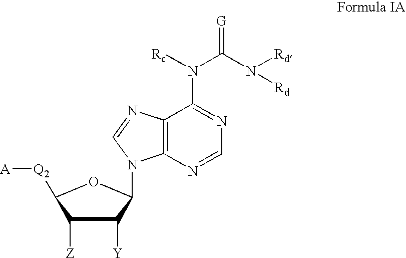
wherein Q₂=C₁₋₈ alkyl, C₂₋₈ alkenyl, C₂₋₈ alkynyl, optionally containing one or more heteroatoms, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ heterocycle, —(CO)—, or absent;
A is H, —OR, —SR, —C₁₋₃ alkyl, C₂₋₃ alkenyl, or C₂₋₃ alkynyl, and each hydrogen of said alkyl or alkenyl is optionally substituted by 0 to 2 fluorine groups, 0 to 1 methyl group, 0 to 2—[(CO)OR] groups, or 0 to 1 —(OR) group;
R_{c}=H;
G=O; and
R_{d} and R_{d′} are independently selected from the group consisting of: H, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, and C₂₋₆ heterocycle, where all rings or chains optionally bear one or more desired substituents; or
R_{d} and R_{d′} groups are taken together to form a ring of 4 to 7 members, with or without unsaturation and with or without heteroatoms in place of ring-carbon units;
Y=H, OH, or ORₐ;
Z=H, OH, or OR_{b}; with the proviso that Y and Z are not both H;
R is selected from the group consisting of: H, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, and C₂₋₆ heterocycle; where all rings or chains optionally bear one or more desired substituents;
Rₐ and R_{b} are residues which are linked directly to the 2′ and/or 3′ oxygens of the furanose via a carbon atom according to Formula III, or linked directly to the two 2′ and 3′ oxygens of the furanose via a common carbon atom according to Formula IV; 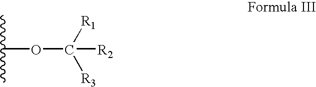
wherein:
O is the corresponding 2′ and/or 3′ oxygen of the furanose;
R₁, R₂, and R₃ are independently H, alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted, such that the moiety defined by Formula III is an ether; or
R₁ and R₂ are independently H, alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted; and R₃ is alkoxy, cycloalkoxy, aralkyloxy, aryloxy, substituted aralkyloxy, or substituted aryloxy, such that the moiety defined by Formula III is an acyclic acetal or ketal; or
R₁ and R₂ are taken together as oxygen or sulfur doubly bonded to C₁ and R₃ is alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted, such that the moiety defined by Formula III is an ester or thioester; or
R₁ and R₂ are taken together as oxygen or sulfur doubly bonded to C₁ and R₃ is amino or mono- or disubstituted amino, where the substituents are independently alkyl, alkenyl, alkynyl, cycloalkyl, cycloalkenyl, aryl, aralkyl, aralkenyl, aralkynyl, heteroaryl, heteroarylalkenyl, or heteroarylalkynyl, optionally substituted, such that the moiety defined by Formula III is a carbamate or thiocarbamate; or
R₁ and R₂ are taken together as oxygen or sulfur doubly bonded to C₁ and R₃ is alkoxy, cycloalkoxy, aralkyloxy, aryloxy, substituted aralkyloxy, or substituted aryloxy, such that the moiety defined by Formula III is a carbonate or thiocarbonate; or
R₃ is not present and R₁ and R₂ are taken together as oxygen or sulfur doubly bonded to C and both the 2′ and 3′ oxygens of the furanose are directly bound to C to form a cyclical carbonate or thiocarbonate; 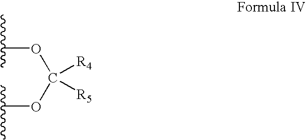
wherein:
O is the 2′ and 3′ oxygens of the furanose; and the 2′ and 3′ oxygens of the furanose are linked by a common carbon atom to form a cyclical acetal, cyclical ketal, or cyclical orthoester;
for the cyclical acetal and ketal, R₄ and R₅ are independently hydrogen, alkyl, alkenyl, alkynyl, cycloalkyl, cycloalkenyl, aryl, aralkyl, aralkenyl, aralkynyl, heteroaryl, heteroarylalkenyl, heteroarylalkynyl, optionally substituted; or
R₄ and R₅ are joined together to form a homocyclic or heterocyclic ring composed of 3 to 8 atoms, preferably 3 to 6 atoms;
for the cyclical orthoester, R₄ is hydrogen, alkyl, alkenyl, alkynyl, cycloalkyl, cycloalkenyl, aryl, aralkyl, aralkenyl, aralkynyl, heteroaryl, heteroarylalkenyl, heteroarylalkynyl, optionally substituted, and R₅ is alkyloxy, cycloalkyloxy, aralkyloxy, aryloxy, substituted aralkyloxy, or substituted aryloxy.

13. The method according to claim 12, wherein said compound is a compound of Formula IB, 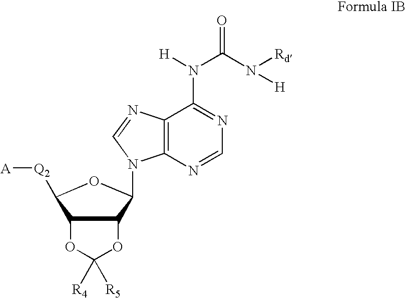
wherein Q₂=C₁₋₈ alkyl, C₂₋₈ alkenyl, C₂₋₈ alkynyl, optionally containing one or more heteroatoms, aralkyl, aralkenyl, aralkynyl, heteroaryl, heteroarylalkenyl, heteroarylalkynyl, C₂₋₆ heterocycle, or absent,
A is —OR, —SR, —COOR;
the first atom of the moiety A/Q₂ directly attached to the 4′ position is C;
R and R_{d′} are independently selected from the group consisting of: H, C₁₋₈ alkyl, C₂₋₈ alkenyl, C₂₋₈ alkynyl, C₃₋₇ cycloalkyl, C₄₋₇ cycloalkenyl, aryl, aralkyl, aralkenyl, aralkynyl, heteroaryl, heteroarylalkenyl, heteroarylalkynyl, C₂₋₆ heterocycle; where all rings or chains optionally bear one or more desired substituents; and
R₄ and R₅ are independently hydrogen, alkyl, alkenyl, alkynyl, cycloalkyl, cycloalkenyl, aryl, aralkyl, aralkenyl, aralkynyl, heteroaryl, heteroarylalkenyl, heteroarylalkynyl, optionally substituted.

14. The method according to claim 13, wherein R_{d′}=C₁₋₈ alkyl, C₂₋₈ alkenyl, aryl, aralkyl, aralkenyl, Q₂=C₁₋₈ alkyl or absent, A=COOH, OH, or COOCH₃, R₄=H, and R₅=aryl, aralkyl, or aralkenyl.

15. The method according to claim 14, wherein said compound is Compound 11:

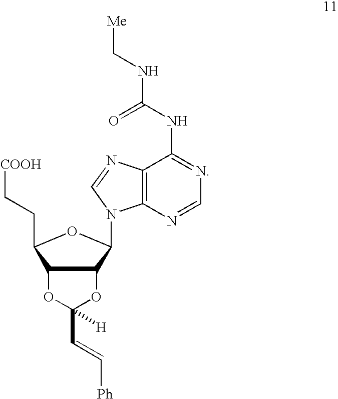

16. The method according to claim 14, wherein said compound is Compound 13, 23, 24, 25, 26, 27, or 28: 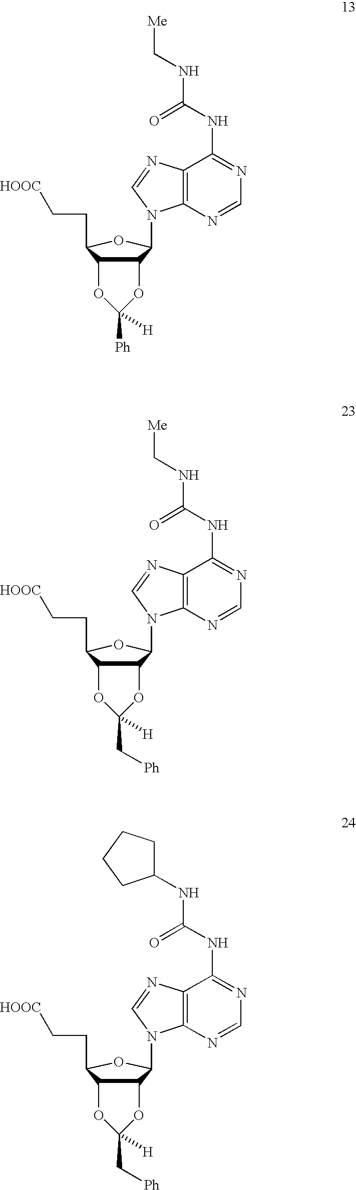 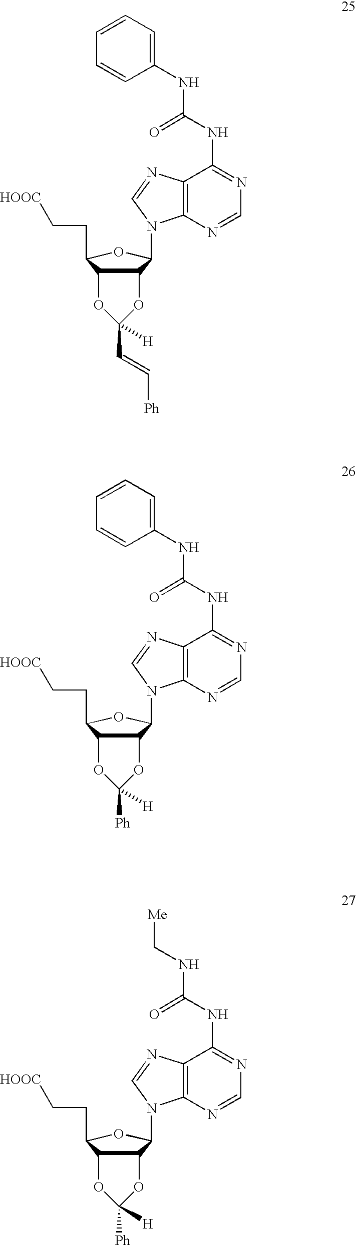 [chemical structure]
